(19) United States
(12) Patent Application Publication
(10) Pub. No.: US 2006/0210619 A1
(43) Pub. Date: Sep. 21, 2006

(54) Limposomes containing asiaticoside and the uses thereof

(76) Inventors: Jianming Chen, Shanghai (CN); Luo Lu, Shanghai (CN); Shen Gao, Shanghai (CN); Huifen Lin, Shanghai (CN); Shaomin Wei, Shanghai (CN); Yangmei Zhang, Shanghai (CN); Huiliang Li, Shanghai (CN); Yanqiang Zhong, Shanghai (CN); Qing Shi, Shanghai (CN); Yiquang Guo, Shanghai (CN); Fei Guan, Shanghai (CN); Wei Wang, Shanghai (CN); Laiji Ma, Shanghai (CN); Juan Gu, Shanghai (CN)

Correspondence Address: BUTZEL LONG, 350 SOUTH MAIN STREET, SUITE 300, ANN ARBOR, MI, 48104, US

(21) Appl. No.: 10/544,088

(22) PCT Filed: Jan. 30, 2004
(86) PCT No.: PCT/CN04/00086

§ 371 Date: Jan. 10, 2006

(30) Foreign Application Priority Data
Jan. 30, 2003 (CN) 03115296.1

(51) Int. Cl. A61K 9/127; A61K 31/704

(52) U.S. Cl. 424/450; 514/26; 514/33

(57) ABSTRACT

This invention belongs to the chemical field, which is related to the fields of pharmaceutical preparations and cosmetic, especially to asiaticoside-liposome and its use for preparing pharmaceutical preparations and cosmetic. In this invention, asiaticoside and lipid components are fused by heating or dissolved in organic solvents, then treated with the rotary thin layer evaporation technique, hydrated by adding aqueous solution under shaking to afford lipid dispersing aqueous solution, and then treated by using the technics of sonication, homogeneous emulsification, microjet and extruding filtration to enwrap asiaticoside in the middle of liposomal bilayer membranes to form the hydrophilic asiaticoside-liposome. Asiaticoside-liposome of this invention can enhance the stability, skin penetrability and hydrophilicity of asiaticoside, it can be used for the preparation of cosmetic and pharmaceutical preparations especially skin-penetrated pharmaceutical preparations, and it is more convenient and easy to prepare skin-penetrated pharmaceutical preparations and cosmetic containing asiaticoside.

### TECHNICAL FIELD

This invention belongs to the chemical field and is related to the fields of pharmaceutical preparations and cosmetics. More specifically, the present invention is directed to asiaticoside-liposomes and their use in the preparation of pharmaceutical compositions and cosmetics.

### BACKGROUND TECHNOLOGY

Centella asiatica(L.) Urban belongs to the Umbellifera family. Its herb can be used as an officinal, which has the effects of defervescence, diuretic, detoxicating, anti-swelling, etc. As a folk medicine in China, the extract of Centella asiatica is used as a remedy for jaundice with damp-heat pathogen, wounds, dermal ulcers, etc. Existing data indicates that the component of triterpene saponins extracted from Centella asiatica can distinctly facilitate the wound healing process, stimulate the growth of granulation, promote keratinization of the epidermis, and redound to allow generation of new connective tissue. In addition, the component of triterpene saponins extracted from Centella asiatica can also be used as a remedy for burns, lower limb ulcers, wounds, adhesion of tendons, etc. Moreover, asiaticoside shows significant activity for scar-hyperplasia and keloid, and it can prevent skin from erythema induced by ultraviolet irradiation. Therefore much interest exists for developing asiaticoside into functional cosmetics that can prevent and cure cutaneous diseases.

Asiaticoside is a triterpene saponin. Attempts at practical use find that asiaticoside can hardly permeate skin because of its big molecular weight (approximate 936), bad liposolubility and water-solubility. In addition, asiaticoside is instable in air and solutions and can easy to be oxidized and degraded because of the character of its structure. These factors influence the ability to prepare stable pharmaceutical preparations and cosmetics. Moreover, bad liposolubility and water-solubility result in difficulties with the preparation process because asiaticoside can not be mixed with other components of pharmaceutical and cosmetic compositions and formulations. These disadvantageous factors restrict the further development and the application of asiaticoside in the field of pharmaceutical compositions and formulations that are intended to be administered per cutem and cosmetic compositions and formulations. Therefore, a need exists to find a suitable drug-carrier which can enhance the chemical stability and skin penetrability of asiaticoside so as to be convenient for the preparation of its pharmaceutical preparations and cosmetic.

### DISCLOSURE OF THE INVENTION

One aspect of the present invention is to provide asiaticoside-liposomes for skin use to overcome the previous inability to use asiaticoside in pharmaceutical preparations that are intended to be administered per cutem and cosmetics.

Another aspect of the present invention is to provide for the use of asiaticoside-liposomes for preparing pharmaceutical compositions and formulations and cosmetics which contain asiaticoside.

### BEST MODE FOR CARRYING OUT THE INVENTION

The asiaticoside-liposomes of the present invention are a kind of opalescent suspension. It is just necessary to uniformly mix the asiaticoside-liposomes with the other components when preparing pharmaceutical compositions and formulations and cosmetics. The asiaticoside-liposomes for skin use are hydrophilic opalescent suspensions in which the asiaticoside is enwrapped in the middle of liposome bilayer membranes. The present invention can enhance not only asiaticoside's stability but also its skin penetrability and hydrophilicity, and it is more propitious to prepare pharmaceutical compositions and formulations and cosmetics of asiaticoside according to the present invention.

The asiaticoside-liposomes for skin use provided by the present invention is prepared by the following methods and steps:

1. Asiaticoside monomer is isolated from the total saponins of Centella asiatica according to conventional methods;

2. The asiaticoside and lipid components used in the liposomes compositions and formulations are fused by heating or dissolution in organic solvents to make a lipid solution;

3. The lipid solution is placed into rotary evaporator, then a lipid film is produced at the bottom of the vessel by the rotary thin layer evaporation technique;

4. A lipid dispersing aqueous solution is produced after the lipid film has been hydrated by adding an aqueous solution while shaking the resulting mixture, or by mixing the lipid solution from step 2 with an aqueous solution directly under shaking;

5. The asiaticoside-liposome is obtained after the lipid dispersing aqueous solution has been treated by using the techniques of sonification, homogeneous emulsification, microjet and extruding filtration.

The asiaticoside content in the asiaticoside-liposomes developed for skin use according to the present invention is 0.1˜10%.

In the liposomes compositions and formulation of the present invention, ceramide is included in the liposomal bilayer structure as an active component.

In addition, at least one of the following components should be included in the liposomes: soybean lecithin, yolk lecithin, distearoyl phosphatidylcholine, dipalmitoyl phosphatidylcholine, poloxamer, dimyristoyl phosphatidylcholine, tween, span, nonionic surfactant Brij, bile salt, cholesterol.

In the liposome compositions and formulation of the present invention, asiaticoside and lipid components of the liposomes account for 0.1˜10% and 0.1˜40% respectively.

The organic solvents used according to the present invention include dichlormethane, chloroform, ether, and ethanol.

The aqueous solutions used according to the present invention include distilled water, deionized water, purified water, and phosphate buffer.

A method for the preparation of liposomal emulsions containing ceramide is mentioned in CN 98110614.5 in which drugs carried by the liposomes are provided with stable chemical properties so that they are difficult to oxidize and have the function of skin protection such as moisturizing, preventing drying, desquamating, etc. These drugs can be easily absorbed by the skin. Therefore, the liposomes are suitable as cosmetic additives and drug-carriers for external use. Analogous methods in which liposomes are applied to the preparation of pharmaceutical preparations and cosmetics are disclosed in ZL 96116044.6, CN 96192625.2, and CN 93114073.0.

The asiaticoside-liposomes of the present invention can be applied to the preparation of pharmaceutical compositions and formulations and cosmetics. The asiaticoside-liposomes can be prepared using conventional methods or the methods described in aforementioned patent documents. Forming the asiaticoside-liposomes according to the present invention enhances the stability, skin penetrability and hydrophilicity of asiaticoside so that it is more convenient and suitable to prepare cosmetic or pharmaceutical compositions and formulations containing the asiaticoside.

The asiaticoside-liposomes of the present invention are provided with the following advantages:

1. The asiaticoside has enhanced stability. Drugs are enwrapped in the middle of liposomal bilayers which can prevent the drugs from being destructed by instable factors such as light, oxygen, acid, base and so on. As a consequence, the stability of the drugs is enhanced. It has been determined that the liposomes can enhance the stability of drugs in both in vitro and in vivo applications and prolong action time of drugs in in vivo applications.

2. The asiaticoside has enhanced skin penetrability. Liposomes are drug carriers that are composed of lipid bilayers which have more comparability and compatibility with biological tissue, and can enhance skin penetrability of drugs. Liposomes not only enhance skin penetrability of drugs, but also retain larger quantity of drugs between epidermis and dermis however, the dosage entering into the hematological system is decreased, so that general adverse effects can be efficiently avoided. Liposomes can enhance the skin penetrability of drugs by the mechanism of hydration, fusion, penetration, etc. Furthermore, plentiful ceramides are contained in stratum comeum of human skin. According to similarity-compatibility theory, liposomes containing ceramides in lipid bilayers can further enhance skin penetrability and absorbability or drugs. The asiaticoside-liposomes of the present invention contain ceramides in the lipid bilayers which allows them to further enhance the skin penetrability of asiaticoside.

3. The asiaticoside-liposomes of the present invention can be mixed discretionarily with other components used in compositions and formulations which make it more simple and convenient to prepare pharmaceutical compositions and formulations and cosmetics containing asiaticoside. In compositions and formulations of most cosmetics the ground substance is hydrophilic or emulsive. Thus, components of the compositions and formulations should be hydrophilic or lipophilic. It is difficult to prepare cosmetics containing asiaticoside because asaiticoside has bad hydrophilicity and lipophilicify. Liposomes are a kind of drug carrier with high hydrophilicity, by which asiaticoside is encapsulated and the hydrophilicity of the drug is thereby enhanced. The encapsulated drug can then be mixed discretionarily with other components of the compositions and formulations. It is more simple and convenient to prepare pharmaceutical compositions and formulations and cosmetics containing asiaticoside.

### DETAILED EXAMPLES

### Example 1

30 g asiaticoside, 20 g soybean lecithin, 30 g cholesterol, 40 g poloxamer F₆₈, 10 g ceramide, 200 ml chloroform, 100 ml ethanol and 1000 ml phosphate buffer (pH 7.4) were placed into a 1000 ml round bottom flask, and dissolved in a solution of chloroform and ethanol. The resulting mixture was subject to a rotary thin layer evaporation technique in a thermostatic waterbath at a temperature of 25˜40° C. so that a lipid film was formed at the bottom of the flask. Then, 800 ml phosphate buffer (pH 7.4) was added to flask. After the lipid film was hydrated under shaking, phosphate buffer (pH 7.4) was added to the mixed solution to produce a volume of 1000 ml. Thereafter asiaticoside-liposome was produced after sonification (output 4, duty cycle 50%, time 20 mins).

### Example 2

50 g asiaticoside, 50 g yolk lecithin, 50 g cholesterol, 20 g ceramide and 1000 ml phosphate buffer (pH 7.4) were placed into a conical flask and fused by heating or dissolved in organic solvent to produce a lipid solution that was placed in a thermostatic waterbath at a temperature of 80° C. 800 ml phosphate buffer (pH 7.4) was placed in a waterbath till its temperature was the same as the temperature of the lipid solution. Then an aqueous solution and the lipid solution were mixed together while shaking the mixture which was then cooled. Phosphate buffer (pH 7.4) was added to the mixed solution to produce a volume of 1000 ml. After homogenizing 6 times using a high pressure homogenization technique (higher pressure: 60 MPa, lower pressure: 10 MPa), asiaticoside-liposome was produced.

### Example 3

20 g asiaticoside, 20 g dipalmitoyl phosphatidylcholine, 30 g poly-dioxyvinylcetylether, 40 g cholesterol, 40 g ceramide, 200 ml dichloromethane, 200 ml ethanol and 100 ml phosphate buffer (pH 7.4) were placed into a 1000 ml round bottom and dissolved in a mixed solution of dichloromethane and ethanol by heating. The resulting mixture was subjected to a thin layer evaporation technique in a thermostatic waterbath at a temperature of 25˜40° C., to produce a lipid film at the bottom of the flask. Then, 800 ml phosphate buffer (pH 7.4) was added to the flask. After the lipid film was hydrated under shaking, phosphate buffer (pH 7.4) was added to the mixed solution to produce a volume of 1000 ml. The mixed solution was filtrated extrudedly from poly-(carbonic acid fibrous tunic) and then asiaticoside-liposome was obtained.

### Example 4

Stability Experiment

Samples of each of the asiaticoside-liposome products produced in Examples 1-3 and an asiaticoside aqueous solution were placed airtight containers at a temperature of 40° C., and a relative humidity 75%. The content of asiaticoside in asiaticoside-liposome samples and the asiaticoside aqueous solution was determined by HPLC after 0, 1, 2, 3 months. The content of asiaticoside in asiaticoside-liposome samples and the asiaticoside aqueous solution was assumed to be 100% at 0 month. The content of asiaticoside at other times was obtained comparing with it at 0 month, then the percentage that the amount of drug changed with time was obtained. The result indicated that after placed for three months at a temperature of 40° C., and a relative humidity 75%, the content of asiaticoside in asiaticoside-liposome samples changed a little, but the content of asiaticoside in the asiaticoside aqueous solution had decreased. This proves that asiaticoside encapsulated by liposomes could enhance drug stability.

Table 1 was the comparison of asiatoside's stability in liposomes and aqueous solution.

TABLE 1
The variety percentage of asiacoside's content (%)

Time (month)

| | 0 | 1 | 2 | 3 |
|---|---|---|---|---|
| Liposomes | 100.00 | 87.56 | 75.41 | 68.02 |
| Aqueous solution | 100.00 | 99.52 | 98.69 | 98.12 |

n = 3

1. An asiaticoside-liposome composition which comprises: an asiaticoside that is enwrapped in the middle of liposomal bilayer membranes to form a hydrophilic opalescent suspension; and ceramide that is included in the liposomal bilayer membrane structure as an active component.

2. method of producing an asiaticoside-liposome composition which comprises the steps of:
a) providing asiaticoside and a liposome;
b) fusing the asiaticoside and lipid components of the liposome by heating or dissolved in an organic solvent to produce a lipid solution;
c) subjecting the lipid solution to an evaporation technique to produce a lipid film;
d) hydrating the lipid film to produce dispersed lipid aqueous solution;
e) subjecting the dispersed lipid aqueous solution to at least one of sonication, homogeneous emulsification, microjet and extruding filtration to produce an asiaticoside.

3. An asiaticoside-liposome composition according to claim 1, further comprising at least one of soybean lecithin, yolk lecithin, distearoyl phosphatidylcholine, dipalmitoyl phosphatidylcholine, poloxamer, dimyristoyl phosphatidyl choline, tween, span, nonionic surfactant Brij, bile salt, and cholesterol.

4. An asiaticoside-liposome composition according to claim 1, wherein the asiaticoside and lipid components of the asiaticoside-liposome composition account for 0.1˜10% and 0.1˜40% of the composition respectively.

5. The method of claim 2, wherein the organic solvent comprises at least one of dichloromethane, chloroform, ether and ethanol.

6. The method of claim 2, wherein the said aqueous solution comprises at least one of distilled water, deionized water, purified water, and phosphate buffer.

7. An asiaticoside-liposome composition according to claim 1, which comprises a pharmaceutical composition.

8. An asiaticoside-liposome composition according to claim 1, which comprises a skin-penetrable pharmaceutical composition.

9. An asiaticoside-liposome composition according to claim 1, which comprises a cosmetic.

10. A method of producing an asiaticoside-liposome composition which comprises the steps of:
a) providing asiaticoside and a liposome;
b) fusing the asiaticoside and lipid components of the liposome by heating or dissolved in an organic solvent to produce a lipid solution;
c) mixing lipid solution with an aqueous solution to produce a dispersed lipid aqueous solution;
e) subjecting the dispersed lipid aqueous solution to at least one of sonication, homogeneous emulsification, microjet and extruding filtration to produce an asiaticoside.

11. The method of claim 2, wherein the asiaticoside and lipid components of the asiaticoside-liposome composition account for 0.1˜10% and 0.1˜40% of the composition respectively.

12. The method of claim 10, wherein the asiaticoside and lipid components of the asiaticoside-liposome composition account for 0.1˜10% and 0.1˜40% of the composition respectively.

13. The method of claim 10, wherein the organic solvent comprises at least one of dichloromethane, chloroform, ether and ethanol.

14. The method of claim 10, wherein the said aqueous solution comprises at least one of distilled water, deionized water, purified water, and phosphate buffer.

15. The method of claim 2, wherein the asiaticoside-liposome composition comprises a pharmaceutical composition.

16. The method of claim 2, wherein the asiaticoside-liposome composition comprises a skin-penetrable pharmaceutical composition.

17. The method of claim 2, wherein the asiaticoside-liposome composition comprises a cosmetic.

18. The method of claim 10, wherein the asiaticoside-liposome composition comprises a pharmaceutical composition.

19. The method of claim 10, wherein the asiaticoside-liposome composition comprises a skin-penetrable pharmaceutical composition.

20. The method of claim 10, wherein the asiaticoside-liposome composition comprises a cosmetic.
